(19) United States
(12) Patent Application Publication
(10) Pub. No.: US 2003/0148468 A1
(43) Pub. Date: Aug. 7, 2003

(54) Method of preparing antibody by gene immunization

(76) Inventors: Seishi Kato, Kanagawa (JP); Naoki Nagata, Kanagawa (JP); Naoko Fujimura, Kanagawa (JP); Midori Kobayashi, Kanagawa (JP); Koichi Ito, Kanagawa (JP); Yoshiko Ishizuka, Kanagawa (JP)

Correspondence Address: WENDEROTH, LIND & PONACK, L.L.P., 2033 K STREET N. W., SUITE 800, WASHINGTON, DC, 20006-1021, US

(21) Appl. No.: 10/088,859

(22) PCT Filed: Jul. 24, 2001
(86) PCT No.: PCT/JP01/06371

(30) Foreign Application Priority Data
Jul. 24, 2000 (JP) 2000-222743
Aug. 24, 2000 (JP) 2000-254407

(51) Int. Cl. C12P 21/04; C12N 15/00

(52) U.S. Cl. 435/70.21; 435/320.1

(57) ABSTRACT

The present invention of the application provides a method for producing an antibody which comprises inoculating an expression vector expressing a fusion protein to an animal, and isolating and purifying an antibody against an antigenic protein from the animal, wherein the fusion protein is an antigenic protein fused to the C-terminal side of a transmembrane domain in which the N-terminal side is located in the cell and the C-terminal is out of the cell. According to the present invention, an antibody against an antigenic protein, which it was difficult to produce in the so far known gene immunization, can be produced. The resulting an antibody is useful as drugs, diagnostic agents, and reagents for the research.

### TECHNICAL FIELD

The invention of the present application relates to a method for producing an antibody by gene immunization. More specifically, the invention relates to a method of enabling easy production of an antibody useful as drugs, diagnostic agents, reagents for the research, and etc., and to an expression vector used in this method.

### BACKGROUND ART

An antibody has widely been utilized as reagents for the research for the purpose of detection, purification, elimination, inhibition of a protein or the like, because it has property of recognizing- specific protein and binding thereto. Recently, it has widely been used not only as reagents for the research but also as drugs or diagnostic agents.

In producing antibodies, it has so far been general to use a method that a large amount of protein as an antigen is purified and injected to an animal or animals such as rabbits or mice to collect antibodies generated in sera. It required, however, much time and a great deal of labor to obtain a large amount of a purified antigenic protein. It is desired to provide a more convenient method for producing antibodies, accordingly.

Recently, it was reported that when a gene coding for an influenza virus nucleoprotein is integrated into an expression vector and intramuscularly injected directly as DNA to mice, then virus proteins are produced in the murine bodies and additionally the antibody against these proteins are generated in the sera. (Ulmer et al., Science 259: 1745-1749, 1993; Ginsbert et al., “Vaccines 93”). As a result, this expression vector received much attention as a new type of vaccine, that is, DNA vaccine, since mice have acquired immunity to virus. Thus, it has been designated as gene immunization that an expression vector for an antigenic protein is inoculated directly to an animal to generate immunity. In using gene immunization, however, in some cases, the titer of the generated antibody is very low or no antibody is generated depending on the kind of the antigen used.

It was reported as an example of gene immunization that ovalbumin was fused in the downstream of transmembrane domain of transferrin receptor to form a membrane type and it was injected intramuscularly or subcutaneously to mice in order to investigate an effect of the expression site of antigenic protein on the efficacy of gene immunization. The titer of the antibodies generated, however, rather decreased since the protein was converted into a membrane type. (Boyle et al., Int. Immunol. 9: 1897-1906, 1997).

The purpose of the invention of present application is to provide a method for producing antibodies to proteins, which it was difficult to produce in so far known gene immunization methods.

Additionally, the purpose of the invention is to provide an expression vector used in the above-mentioned method for producing an antibody.

### DISCLOSURE OF THE INVENTION

The present application, as the invention for solving the above-described problems, provides a method for producing an antibody which comprises inoculating an expression vector expressing a fusion protein to an animal, isolating an antibody against an antigenic protein from the animal and purifying the antibody, wherein the fusion protein is an antigenic protein fused with the C-terminal of a transmembrane domain of which the N-terminal side is located in the cell and the C-terminal side is out of the cell.

In a preferred embodiment in this method for producing an antibody, the transmembrane domain is a polypeptide having at least the amino acid sequence from 1st to 26th of SEQ ID NO. 2.

The application also provides an expression vector expressing a fusion protein in which an antigenic protein is fused with the C-terminal side of transmembrane domain of which the N-terminal side is located in the cell and the C-terminal side is out of the cell.

In a preferred embodiment of this expression vector, the transmembrane domain is a polypeptide having at least the amino acid sequence from 1st to 26th of SEQ ID NO. 2.

### BRIEF DESCRIPTION OF THE DRAWINGS

FIG. 1 shows the structure of urokinase fusion gene.

FIG. 2 is an example illustrative of determination of antibody titer by ELISA when gene immunization has been carried out with a urokinase expression vector.

FIG. 3 is an example illustrative of determination of antibody titer by ELISA when gene immunization has been carried out with a nuclear protein HP10496 expression vector.

FIG. 4 shows the respective N-terminal amino acid sequences of fusion proteins comprising urokinase and transmembrane domains in a variety of membrane proteins.

### BEST MODE FOR CARRYING OUT THE INVENTION

In a method for producing antibodies according to the invention, the expression vector to be inoculated to animals may be constructed as an expression vector having a fusion polynucleotide that consists of a polynucleotide encoding an antigenic protein and a polynucleotide encoding a transmembrane domain.

As for an antigenic protein, any one that can generate an antigen-antibody reaction in vivo may be used. The polynucleotide encoding an antigenic protein may be any one of genomic DNA, cDNA, synthetic DNA, etc., as far as it has an open reading frame (ORF). When the antigenic protein is an inherent secretory protein, it is used after removal of the signal sequence peptide originally possessed by the protein.

As for the transmembrane domain, any domain may be used as far as its N-terminal side is in the cell and the C-terminal side is out of the cell. For example, transmembrane domains of type II-membrane proteins or those of multispan-type membrane proteins may be used. The proteins that an antigenic protein is fused to the C-terminal side of these transmembrane domains take forms that the antigenic protein portion exists on the surface of the cell membrane. As for the transmembrane domain, for example, that of human type-II membrane protein HP10085 (SEQ ID NO: 2) may be used. In this case, the transmembrane domain to be fused with an antigenic protein is a polypeptide containing at least 1st methionine (Met) to 26th lysine (Lys) of SEQ ID NO: 2. The polynucleotide encoding the polypeptide contains at least the base sequence from 151st-228th of SEQ ID NO: 1 (cDNA of human type-II membrane protein HP10085). This polynucleotide is linked to a polynucleotide encoding the above-mentioned antigenic protein, and the expression vector can be constructed using the fusion polynucleotide which expresses a fusion protein that an antigenic protein is fused to the C-terminal side of the transmembrane domain (polypeptide).

As for vectors expressing fusion proteins, any ones for eucaryotic cells may be used as far as they contain a promoter, a splicing region, a poly (A) addition site, etc., such as pKA1, pCDM8, pSVK3, pMSG, pSVL, pBK-CMV, pBK-RSV, EBV vectors, and the like. The two above-described polynucleotides are cloned to these vectors to make expression vectors that express respectively coded proteins as fusion proteins.

Animals to which an expression vector is inoculated include mammals such as mouse, rat, rabbit, etc., and birds such as chicken, etc., which are generally used in producing antibodies. Inoculation of the expression vectors to animals may preferably be achieved using a gene gun technology and the like. When a gene gun technology is used, the expression vector is adsorbed on gold particles and emitted as a jet to the skin with gas pressure and the like for inoculation. The amount of the expression vector to be inoculated is variable depending on the species of animal and may preferably be in a range of 0.1 μg-1 mg/animal. One-shot inoculation is acceptable, but it is desirable to carry out the inoculation twice or more at regular intervals in order to assure generation of the antibodies.

The generation of the antibody may be confirmed by collecting blood, separating serum, and examining the binding reaction with the antigenic protein. For example, a known method such as enzyme immunoassay (ELISA), Western blotting, immuno-precipitation, antibody staining, and the like may be used. After confirmation of the presence of the antibody in the serum by these methods, the serum may be used as a polyclonal antibody specimen as it is or may be purified by affinity column chromatography to yield IgG. Alternatively, the spleen may be taken out from the animal acquiring immunity and the monoclonal antibody can be produced in a conventional manner.

### EXAMPLES

The following examples serve to illustrate the invention in more detail and specifically but are not intended as a limitation thereof. In these examples, basic procedures for recombination of DNA and enzyme reactions are carried out according to the articles, “Molecular Cloning; A laboratory manual”, Cold Spring Harbor Laboratory, 1989. Restriction enzymes and a variety of modified enzymes were obtained from Takara Shuzo Co., Ltd., unless otherwise stated. The compositions of buffer solutions in respective enzyme reactions and the reaction conditions were set according to the specification attached.

(1) Construction of an Expression Vector for the Urokinase-Fusion Protein

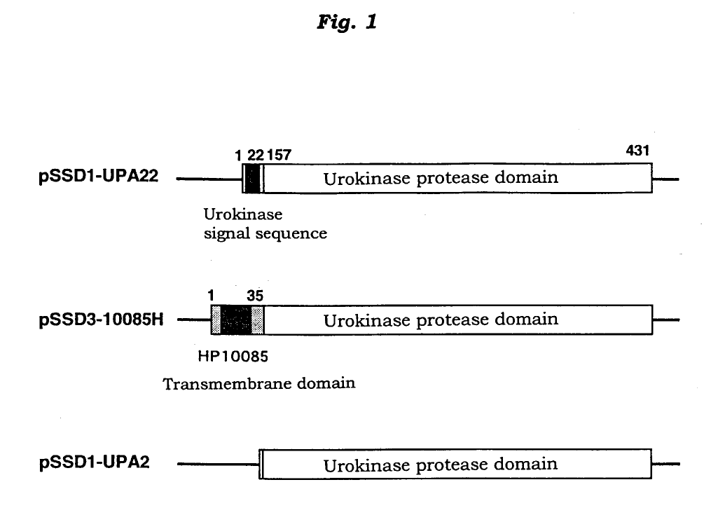

When urokinase is used as an antigenic protein, 3 kinds of expression vectors were used, that is, for secretion expression, for membrane form expression, and for intracellular expression. That is, the following vectors were respectively used: for secretion expression, pSSD1-UPA22 which expresses the signal sequence and protease domain of urokinase (Yokoyama-Kobayashi et al., Gene 163: 193-196, 1995); for membrane form expression, pSSD3-10085H which expresses a protein prepared by fusing a sequence from the N-terminal side to the 35th proline (Pro) of type II-membrane protein HP10085 (SEQ ID NO: 2) with the protease domain of urokinase (Yokoyama-Kobayashi et al., Gene 228: 161-167, 1999); for intracellular expression, pSSD1-UPA2 which expresses only the protease domain of urokinase by eliminating the signal sequence of urokinase (Yokoyama-Kobayashi et al., Gene 163: 193-196, 1995). FIG. 1 shows the structure of respective fusion gene portions. In every case, the structure is the same except the portion encoding fusion protein. That is, they have the early promoter of SV40, 16S mRNA splicing region of SV40 and poly (A) addition site of SV40.

(2) Construction of an Expression Vector for the Fusion Protein with Nuclear Protein

As an antigenic protein, protein HP10496 localized in the nuclear spliceosome was used. cDNA clone pHP10496 encoding this protein was cloned from a human gastric cancer cDNA library (WO98/21328). This has the base sequence of SEQ ID NO: 3 as well as ORF which encodes protein HP10496 comprising the amino acid sequence of SEQ ID NO: 4. Using a 30 mer sense primer (oligonucleotide of SEQ ID NO: 5) starting from a translation initiation codon to which a EcoRV recognition site was added and a 30 mer antisense primer (oligonucleotide of SEQ ID NO: 6) containing a stop codon to which an EcoRV recognition site was added, the translation region was amplified by PCR using pHP10496 as template. The PCR product was digested with EcoRV, and inserted into an EcoRV-NotI (blunt ends) site of a membrane-type of urokinase expression vector pSSD3-10085N to construct a fusion gene expression vector pHP10085N-10496.

In order to obtain an antigenic protein for antibody detection, an expression vector for expressing a protein fused with glutathione-S-transferase (GST) in Escherichia coli was constructed. Using a 28 mer sense primer (oligonucleotide of SEQ ID NO: 7) starting from a translation initiation codon to which a EcoRI recognition site was added and a 32 mer antisense primer (oligonucleotide of SEQ ID NO: 8) containing a stop codon to which a SalI recognition site was added, the translation region was amplified by PCR using pHP10496 as template. The PCR product was digested with EcoRI and SalI and inserted into the EcoRI-SalI site of pGEX-5X-1 (Pharmacia Corp.). After confirmation of the sequence, a host Escherichia coli BL21 was transformed and incubated on an LB medium at 37° C. for 5 hours. Then, IPTG was added at a final concentration of 0.4 mM, and the mixture was further incubated at 37° C. for 2.5 hours. The cells were centrifuged, dissolved in a lysis buffer solution (50 mM Tris-HCl (pH7.5), 1 mM EDTA, 1% Triton X-100, 0.2% SDS, 0.2 mM PMSF), frozen once at −80° C. and thawed, and destroyed by sonication. After centrifugation at 1000×g for 30 minutes, Glutathione Sepharose 4B was added to the supernatant, and the mixture was incubated at 420 C. for 1 hour. The beads were washed well, and the fusion protein was eluted with an elution buffer solution (10 mM Tris-HCl, 50 mM glutathione). As a result, fusion protein GST-HP10496 having molecular weight of approximately 47 kDa was obtained.

(3) Gene Immunization

Gene immunization was achieved with a gene gun (Helios Gene Gun System; Japan Bio-Rad Laboratories). Expression vector plasmid DNA was attached onto gold particles according to the protocol. DNA-coated gold particles corresponding to 2 μg of plasmid DNA were shot into the skin of inguinal region of 3 mice (BALB/c) per sample. The immunization was carried out twice a week for 4 weeks, and the blood was then collected.

(4) Detection of the Antibody by ELISA

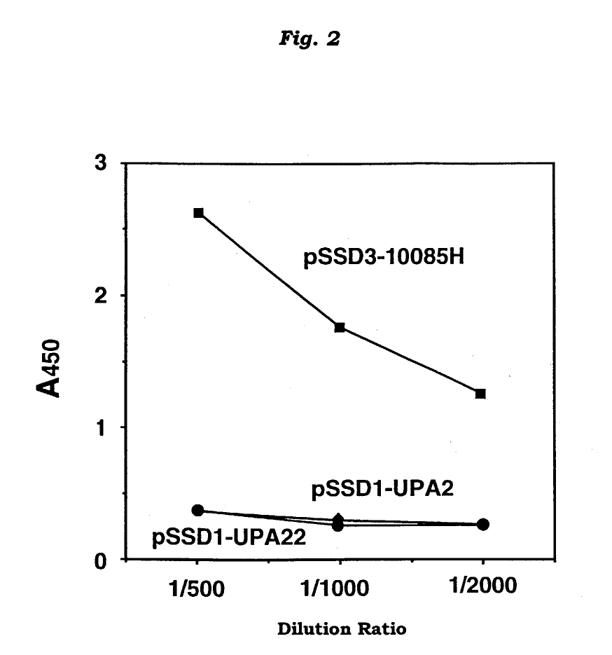

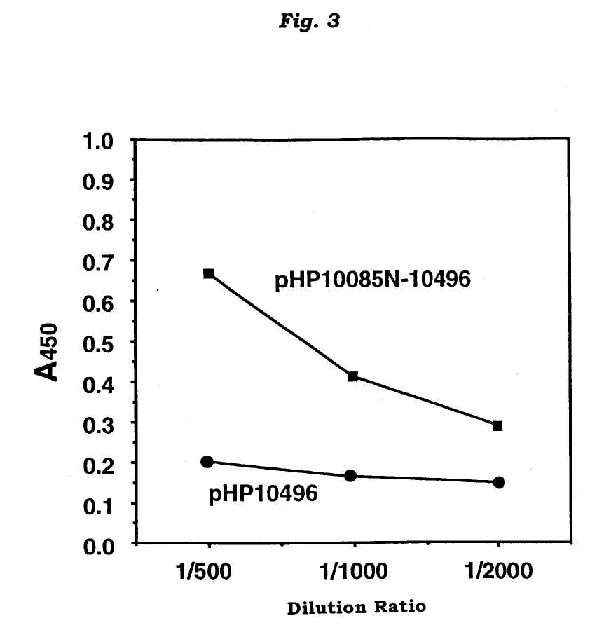

As an antigenic protein, commercially available urokinase (Wakamori Jun-yaku) or GST-HP10496 expressed in Escherichia coli was coated on a plate and it was used in ELISA. The serum obtained by gene immunization with urokinase or nuclear protein HP10496 as antigen was used to measure the titer of the antibodies. The results are shown in FIGS. 2 and 3. In any cases, production of antibodies was recognized only in immunization with the vector expressing the proteins to which the N-terminal of HP10085 was fused.

(5) Gene Immunization with Fusion Proteins Comprising the Transmembrane Domain of Various Species of Type II-Membrane Proteins and Urokinase

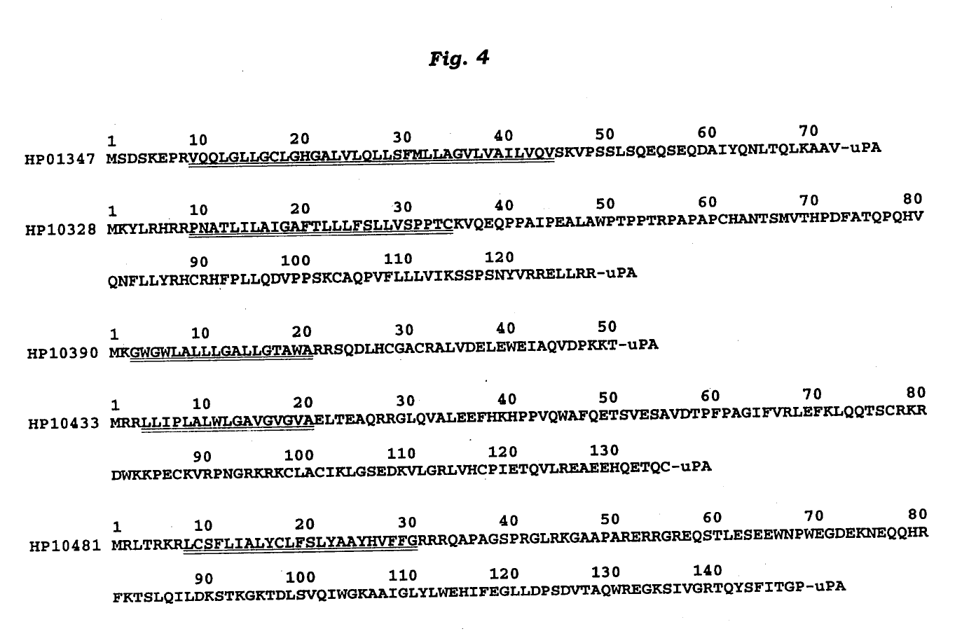

From a human full-length cDNA data bank, the following 5 species of type II-membrane protein cDNAs, HP01347 (SEQ ID NO: 9), HP10328 (SEQ ID NO: 10), HP10390 (SEQ ID NO: 11), HP10433 (SEQ ID NO: 12), and HP10481 (SEQ ID NO: 13) were identified, and the vectors expressing fusion proteins of urokinase with the respective transmembrane domains were constructed (Yokoyama-Kobayashi et al., Gene 228: 161-167, 1999). FIG. 4 shows the respective N-terminal amino acid sequences.

The gene immunization was carried out with these expression vectors according to the method as described in the above item (3), and the antibodies to urokinase were detected according to the method as described in the above item (4). In respective cases, production of antibodies was recognized in the same as in HP10085.

### Industrial Applicability

According to the present invention, an antibody against an antigenic protein, which it was difficult to produce in the so far known gene immunization, can be produced. The resulting an antibody is useful as drugs, diagnostic agents, and reagents for the research.

1. A method for producing an antibody which comprises inoculating an expression vector expressing a fusion protein to an animal, isolating an antibody against an antigenic protein from the animal and purifying the antibody, wherein the fusion protein is an antigenic protein fused with the C-terminal of a transmembrane domain of which the N-terminal side is located in the cell and the C-terminal side is out of the cell.

2. The method for producing an antibody of claim 1, wherein the transmembrane domain is a polypeptide having at least the amino acid sequence from 1st to 26th of SEQ ID NO. 2.

3. An expression vector expressing a fusion protein in which an antigenic protein is fused with the C-terminal of transmembrane domain of which the N-terminal side is located in the cell and the C-terminal side is out of the cell.

4. The expression vector of claim 3, wherein the transmembrane domain is a polypeptide having at least the amino acid sequence from 1st to 26th of SEQ ID NO. 2.


SEQUENCE LISTING (13 sequences)

SEQ ID NO: 1 - DNA, 697 bp, Homo sapiens
  CDS (151)..(600)
tatacctctagtttggagctgtgctgtaaaaacaagagtaacatttttatattaaagttaaataaagttacaactttgaagagagtttctgcaagacatgacacaaagctgctagcagaaaatcaaaacgctgattaaaagaagcacggtatgatgaccaaacataaaaagtgttttataattgttggtgttttaataacaactaatattattactctgatagttaaactaactcgagattctcagagtttatgcccctatgattggattggtttccaaaacaaatgctattatttctctaaagaagaaggagattggaattcaagtaaatacaactgttccactcaacatgccgacctaactataattgacaacatagaagaaatgaattttcttaggcggtataaatgcagttctgatcactggattggactgaagatggcaaaaaatcgaacaggacaatgggtagatggagctacatttaccaaatcgtttggcatgagagggagtgaaggatgtgcctacctcagcgatgatggtgcagcaacagctagatgttacaccgaaagaaaatggatttgcaggaaaagaatacactaagttaatgtctaagataatggggaaaatagaaaataacattattaagtgtaaaaccagcaaagtacttttttaattaaacaaagttcgagttttgtac

SEQ ID NO: 2 - protein, 149 aa, Homo sapiens
MMTKHKKCFIIVGVLITTNIITLIVKLTRDSQSLCPYDWIGFQNKCYYFSKEEGDWNSSKYNCSTQHADLTIIDNIEEMNFLRRYKCSSDHWIGLKMAKNRTGQWVDGATFTKSFGMRGSEGCAYLSDDGAATARCYTERKWICRKRIH

SEQ ID NO: 3 - DNA, 548 bp, Homo sapiens
  CDS (30)..(503)
cttattgctggcggcctgaggagcccatcatggcgacgccccctaagcggcgggcggtggaggccacgggggagaaagtgctgcgctacgagaccttcatcagtgacgtgctgcagcgggacttgcgaaaggtgctggaccatcgagacaaggtatatgagcagctggccaaataccttcaactgagaaatgtcattgagcgactccaggaagctaagcactcggagttatatatgcaggtggatttgggctgtaacttcttcgttgacacagtggtcccagatacttcacgcatctatgtggccctgggatatggttttttcctggagttgacactggcagaagctctcaagttcattgatcgtaagagctctctcctcacagagctcagcaacagcctcaccaaggactccatgaatatcaaagcccatatccacatgttgctagaggggcttagagaactacaaggcctgcagaatttcccagagaagcctcaccattgacttcttccccccatcctcagacattaaagagcctgaatgcctttg

SEQ ID NO: 4 - protein, 157 aa, Homo sapiens
MATPPKRRAVEATGEKVLRYETFISDVLQRDLRKVLDHRDKVYEQLAKYLQLRNVIERLQEAKHSELYMQVDLGCNFFVDTVVPDTSRIYVALGYGFFLELTLAEALKFIDRKSSLLTELSNSLTKDSMNIKAHIHMLLEGLRELQGLQNFPEKPHH

SEQ ID NO: 5 - DNA, 30 bp, Artificial Sequence
  Artificial Sequence Synthesized oligonucleotide
cccgatatctcatggcgacgccccctaagc

SEQ ID NO: 6 - DNA, 30 bp, Artificial Sequence
  Artificial Sequence Synthesized oligonucleotide
cccgatatctcaatggtgaggcttctctgg

SEQ ID NO: 7 - DNA, 28 bp, Artificial Sequence
  Artificial Sequence Synthesized oligonucleotide
cccgaattcatggcgacgccccctaagc

SEQ ID NO: 8 - DNA, 32 bp, Artificial Sequence
  Artificial Sequence Synthesized oligonucleotide
cccgtcgacgcatggtgaggcttctctgggaa

SEQ ID NO: 9 - DNA, 1643 bp, Homo sapiens
  CDS (25)..(915)
aacatctggggacagcgggaaaacatgagtgactccaaggaaccaagggtgcagcagctgggcctcctggggtgtcttggccatggcgccctggtgctgcaactcctctccttcatgctcttggctggggtcctggtggccatccttgtccaagtgtccaaggtccccagctccctaagtcaggaacaatccgagcaagacgcaatctaccagaacctgacccagcttaaagctgcagtgggtgagctctcagagaaatccaagctgcaggagatctaccaggagctgacccagctgaaggctgcagtgggtgagttgccagagaaatccaagctgcaggagatctaccaggagctgacccggctgaaggctgcagtgggtgagttgccagagaaatccaagctgcaggagatctaccaggagctgacccggctgaaggctgcagtgggtgagttgccagagaaatccaagctgcaggagatctaccaggagctgacccggctgaaggctgcagtgggtgagttgccagagaaatccaagctgcaggagatctaccaggagctgacggagctgaaggctgcagtgggtgagttgccagagaaatccaagctgcaggagatctaccaggagctgacccagctgaaggctgcagtgggtgagttgccagaccagtccaagcagcagcaaatctatcaagaactgaccgatttgaagactgcatttgaacgcctgtgccgccactgtcccaaggactggacattcttccaaggaaactgttacttcatgtctaactcccagcggaactggcacgactccgtcaccgcctgccaggaagtgagggcccagctcgtcgtaatcaaaactgctgaggagcagcttccagcggtactggaacagtggagaacccaacaatagcgggaatgaagactgtgcggaatttagtggcagtggctggaacgacaatcgatgtgacgttgacaattactggatctgcaaaaagcccgcagcctgcttcagagacgaatagttgtttccctgctagcctcagcctccattgtggtatagcagaacttcacccacttgtaagccagcgcttcttctctccatccttggaccttcacaaatgccctgagacggttctctgttcgatttttcatcccctatgaacctgggtcttattctgtccttctgatgcctccaagtttccctggtgtagagcttgtgttcttggcccatccttggagctttataagtgacctgagtgggatgcatttagggggcgggcttggtatgttgtatgaatccactctctgttccttttggagattagactatttggattcatgtgtagctgccctgtcccctggggctttatctcatccatgcaaactaccatctgctcaacttccagctacaccccgtgcacccttttgactggggacttgctggttgaaggagctcatcttgcaggctggaagcaccagggaattaattcccccagtcaaccaatggcatccagagagggcatggaggctccatacaacctcttccacccccacatctttctttgtcctatacatgtcttccatttggctgtttctgagttgtagcctttataataaagtggtaaatgttgtaactgc

SEQ ID NO: 10 - DNA, 2186 bp, Homo sapiens
  CDS (118)..(1236)
actctttcttcggctcgcgagctgagaggagcaggtagaggggcagaggcgggactgtcgtctgggggagccgcccaggaggctcctcaggccgaccccagaccctggctggccaggatgaagtatctccggcaccggcggcccaatgccaccctcattctggccatcggcgctttcaccctcctcctcttcagtctgctagtgtcaccacccacctgcaaggtccaggagcagccaccggcgatccccgaggccctggcctggcccactccacccacccgcccagccccggccccgtgccatgccaacacctctatggtcacccacccggacttcgccacgcagccgcagcacgttcagaacttcctcctgtacagacactgccgccactttcccctgctgcaggacgtgcccccctctaagtgcgcgcagccggtcttcctgctgctggtgatcaagtcctcccctagcaactatgtgcgccgcgagctgctgcggcgcacgtggggccgcgagcgcaaggtacggggtttgcagctgcgcctcctcttcctggtgggcacagcctccaacccgcacgaggcccgcaaggtcaaccggctgctggagctggaggcacagactcacggagacatcctgcagtgggacttccacgactccttcttcaacctcacgctcaagcaggtcctgttcttacagtggcaggagacaaggtgcgccaacgccagcttcgtgctcaacggggatgatgacgtctttgcacacacagacaacatggtcttctacctgcaggaccatgaccctggccgccacctcttcgtggggcaactgatccaaaacgtgggccccatccgggctttttggagcaagtactatgtgccagaggtggtgactcagaatgagcggtacccaccctattgtgggggtggtggcttcttgctgtcccgcttcacggccgctgccctgcgccgtgctgcccatgtcttggacatcttccccattgatgatgtcttcctgggtatgtgtctggagcttgagggactgaagcctgcctcccacagcggcatccgcacgtctggcgtgcgggctccatcgcaacacctgtcctcctttgacccctgcttctaccgagacctgctgctggtgcaccgcttcctaccttatgagatgctgctcatgtgggatgcgctgaaccagcccaacctcacctgcggcaatcagacacagatctactgagtcagcatcagggtccccagcctctgggctcctgtttccataggaaggggcgacaccttcctcccaggaagctgagacctttgtggtctgagcataagggagtgccagggaaggtttgaggtttgatgagtgaatattctggctggcgaactcctacacatccttcaaaacccacctggtactgttccagcatcttccctggatggctggaggaactccagaaaatatccatcttctttttgtggctgctaatggcagaagtgcctgtgctagagttccaactgtggatgcatccgtcccgtttgagtcaaagtcttacttccctgctctcacctactcacagacgggatgctaagcagtgcacctgcagtggtttaatggcagataagctccgtctgcagttccaggccagccagaaactcctgtgtccacatagagctgacgtgagaaatatctttcagcccaggagagaggggtcctgatcttaaccctttcctgggtctcagacaactcagaaggttggggggataccagagaggtggtggaataggaccgccccctccttacttgtgggatcaaatgctgtaatggtggaggtgtgggcagaggagggaggcaagtgtcctttgaaagttgtgagagctcagagtttctggggtcctcattaggagcccccatccctgtgttccccaagaattcagagaacagcactggggctggaatgatctttaatgggcccaaggccaacaggcatatgcctcactactgcctggagaagggagagattcaggtcctccagcagcctccctcacccagtatgttttacagattacggggggaccgggtgagccagtgaccccctgcagcccccagcttcaggcctcagtgtctgccagtcaagcttcacaggcattgtgatggggcagccttggggaatataaaattttgtg

SEQ ID NO: 11 - DNA, 814 bp, Homo sapiens
  CDS (145)..(693)
agaatcccggacagccctgctccctgcagccaggtgtagtttcgggagccactggggccaaagtgagagtccagcggtcttccagcgcttgggccacggcggcggccctgggagcagaggtggagcgaccccattacgctaaagatgaaaggctggggttggctggccctgcttctgggggccctgctgggaaccgcctgggctcggaggagccaggatctccactgtggagcatgcagggctctggtggatgaactagaatgggaaattgcccaggtggaccccaagaagaccattcagatgggatctttccggatcaatccagatggcagccagtcagtggtggaggtgccttatgcccgctcagaggcccacctcacagagctgctggaggagatatgtgaccggatgaaggagtatggggaacagattgatccttccacccatcgcaagaactacgtacgtgtagtgggccggaatggagaatccagtgaactggacctacaaggcatccgaatcgactcagatattagcggcaccctcaagtttgcgtgtgagagcattgtggaggaatacgaggatgaactcattgaattcttttcccgagaggctgacaatgttaaagacaaactttgcagtaagcgaacagatctttgtgaccatgccctgcacatatcgcatgatgagctatgaaccactggagcagcccacactggcttgatggatcacccccaggaggggaaaatggtggcaatgccttttatatattatgtttttactgaaattaactgaaaaaatatgaaaccaaaagtac

SEQ ID NO: 12 - DNA, 695 bp, Homo sapiens
  CDS (73)..(564)
aagatttcagctgcgggacggtcaggggagacctccaggcgcagggaaggacggccagggtgacacggaagcatgcgacggctgctgatccctctggccctgtggctgggcgcggtgggcgtgggcgtcgccgagctcacggaagcccagcgccggggcctgcaggtggccctggaggaatttcacaagcacccgcccgtgcagtgggccttccaggagaccagtgtggagagcgccgtggacacgcccttcccagctggaatatttgtgaggctggaatttaagctgcagcagacaagctgccggaagagggactggaagaaacccgagtgcaaagtcaggcccaatgggaggaaacggaaatgcctggcctgcatcaaactgggctctgaggacaaagttctgggccggttggtccactgccccatagagacccaagttctgcgggaggctgaggagcaccaggagacccagtgcctcagggtgcagcgggctggtgaggacccccacagcttctacttccctggacagttcgccttctccaaggccctgccccgcagctaagccagcactgagctgcgtggtgcctccaggaccgctgccggtggtaaccagtggaagaccccagcccccagggagaggaccccgttctatccccagccatgataataaagctgctctcccagctgcctctc

SEQ ID NO: 13 - DNA, 1451 bp, Homo sapiens
  CDS (105)..(1436)
actgcctggaaacgggctgggcctgcctcggacgccgccggtgtcgcggattctctttccgcccgctccatggcggtggatgcctgactggaagcccgagtgggatgcggctgacgcggaagcggctctgctcgtttcttatcgccctgtactgcctattctccctctacgctgcctaccacgtcttcttcgggcgccgccgccaggcgccggccgggtccccgcggggcctcaggaagggggcggcccccgcgcgggagagacgcggccgagaacagtccactttggaaagtgaagaatggaatccttgggaaggagatgaaaaaaatgagcaacaacacagatttaaaactagccttcaaatattagataaatccacgaaaggaaaaacagatctcagtgtacaaatctggggcaaagctgccattggcttgtatctctgggagcatatttttgaaggcttacttgatcccagcgatgtgactgctcaatggagagaaggaaagtcaatcgtaggaagaacacagtacagcttcatcactggtccagctgtaataccagggtacttctccgttgatgtgaataatgtggtactcattttaaatggaagagaaaaagcaaagatcttttatgccacccagtggttactttatgcacaaaatttagtgcaaattcaaaaactccagcatcttgctgttgttttgctcggaaatgaacattgtgataatgagtggataaacccattcctcaaaagaaatggaggcttcgtggagctgcttttcataatatatgacagcccctggattaatgacgtggatgtttttcagtggcctttaggagtagcaacatacaggaattttcctgtggtggaggcaagttggtcaatgctgcatgatgagaggccatatttatgtaatttcttaggaacgatttatgaaaattcatccagacaggcactaatgaacattttgaaaaaagatgggaacgataagctttgttgggtttcagcaagagaacactggcagcctcaggaaacaaatgaaagtcttaagaattaccaagatgccttgcttcagagtgatctcacattgtgcccggtcggagtaaacacagaatgctatcgaatctatgaggcttgctcctatggctccattcctgtggtggaagacgtgatgacagctggcaactgtgggaatacatctgtgcaccacggtgctcctctgcagttactcaagtccatgggtgctccctttatctttatcaagaactggaaggaactccctgctgttttagaaaaagagaaaactataattttacaagaaaaaattgaaagaagaaaaatgttacttcagtggtatcagcacttcaagacagagcttaaaatgaaatttactaatattttagaaagctcatttttaatgaataataaaagttaattatctttttgagct